(19) United States
(12) Plant Patent Application Publication
(10) Pub. No.: US 2025/0386753 P1
(43) Pub. Date: Dec. 18, 2025

(54) Echinacea Plant Named 'BullEchipur 122'

(50) ;

(71) Applicant: Hartwig Bull, Gönnebek (DE)

(72) Inventor: Hartwig Bull, Gönnebek (DE)

(21) Appl. No.: 19/235,231

(22) Filed: Jun. 11, 2025

Related U.S. Application Data
(60) Provisional application No. 63/731,837, filed on Jun. 12, 2024.

(51) Int. Cl. A01H 6/14; A01H 5/02

(52) U.S. Cl. CPC A01H 6/1448; A01H 5/02
USPC PLT/428

(57) ABSTRACT

A new and distinct variety of Echinacea plant named ‘BullEchipur 122’ having red colored flowers, good growth habit, uniform growth habit, good branching habit, and good propagation rates.

### CROSS REFERENCE TO RELATED APPLICATIONS

This application claims the benefit of priority from U.S. Provisional Patent Application No. 63/731,837, filed Jun. 12, 2024, which is incorporated herein by reference in its entirety.

Botanical classification: Echinacea purpurea.

Varietal denomination: ‘BULLECHIPUR 122’.

### BACKGROUND OF THE INVENTION

The present invention relates to a new and distinct cultivar of Echinacea, botanically known as Echinacea purpurea, and hereinafter referred to by the name ‘BullEchipur 122’.

The new Echinacea was discovered by the Inventor in August 2021, in a controlled environment in Gönnebek, Germany, as a part of a planned breeding program. The new Echinacea was selected on the basis of its red colored flowers, good growth habit, uniform growth habit, good branching habit, and good propagation rates.

Asexual propagation of the new cultivar by in vitro culture since September 2021, in Gönnebek, Germany, has shown that the unique features of this new Echinacea are stable and reproduced true to type in successive generations.

### SUMMARY OF THE INVENTION

The following traits have been repeatedly observed and are determined to be unique characteristics of ‘BullEchipur 122’. These characteristics in combination distinguish ‘BullEchipur 122’ as a new and distinct cultivar:
1. Medium compact growth
2. Dark red flowers
3. Lots of flowers
4. More branching habit
5. Very uniform growth
6. Nice dark green leaves
7. Fast crop time

In side-by-side comparisons conducted by the Inventor in Gönnebek, Germany, plants of the new Echinacea differed from plants of the female parent, ‘Bul-2019-26’ of Echinacea purpurea, in the following characteristics:
1. More compact
2. Dark red flower color
3. Good branching
4. Smaller dark green leaves

In side-by-side comparisons conducted by the Inventor in Gönnebek, Germany, plants of the new Echinacea differed from plants of the male parent, ‘Bul-2019-47’ of Echinacea purpurea, in the following characteristics:
1. Taller growths
2. Dark red flower color
3. More branches
4. Lots of flowers

Plants of the new Echinacea can be compared to the plants of the Echinacea cultivar ‘Tomato Soup’, patented as U.S. Plant Pat. No. 19,427 P2. In side-by-side comparisons conducted by the Inventor in Gönnebek, Germany, plants of the new Echinacea differed from plants of the cultivar ‘Tomato Soup’ in the following characteristics:
1. More compact
2. More branching habit
3. Very even habit
4. More flowers
5. Darker red flowers
6. Nice cleaner leaves

### DESCRIPTION OF THE DRAWINGS

The accompanying colored photographs illustrate the overall appearance of the new Echinacea, showing the colors as true as it is reasonably possible to obtain in colored reproductions of this type. Colors in the photographs may differ slightly from the color values cited in the detailed botanical description, which accurately describes the colors of the new Echinacea.

FIG. 1 comprises a top perspective view of a typical plant of ‘BullEchipur 122’ shown under natural lighting.

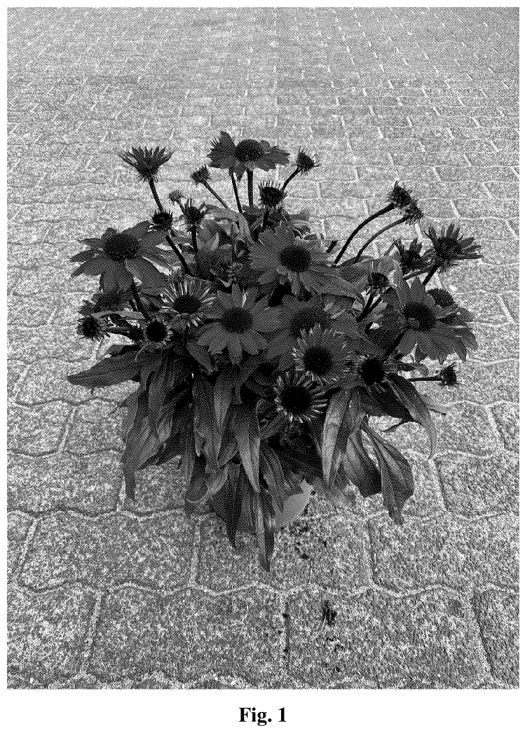

### DESCRIPTION OF THE PLANT

The cultivar Echinacea has not been observed under all possible environmental conditions. The phenotype may vary somewhat with variations in environment such as temperature and light intensity without, however, any variance in genotype.

The aforementioned photographs and following observations and measurements describe plants of the new Echinacea that were grown in an outdoor nursery in Gönnebek, Germany. During the production of the plants, temperatures averaged about 18-20° C. Plants used for the photographs and description were about 23 weeks from planting. Color references are made to The Royal Horticultural Society Colour Chart, 2015 version, except where general terms of ordinary dictionary significance are used.
Plant:
   Botanical classification.—Echinacea purpurea.
   Parentage.—Bul 2019-26 and Bul 2019-47.
   Propagation.—Time to initiate roots: About 7 days. Time to develop roots: About 40-50 days. Time to produce a finished flowering plant: About 12-14 weeks. Root description: Lots of roots, free-branching and healthy-looking root system.
   Plant description.—Growth rate/vigor: Medium, upright. Plant height: 45-60 cm. Plant width: 35-40 cm. Branching habit: Lots of branches, upright, shows a medium compact plant. Lateral branches: Number per plant: 25-35. Length: 4-40 cm. Diameter: 3-8 mm. Angle: Upright, outward. Strength: Strong. Texture: Pubescent. Color: 183A. Internode length: 1-7 cm. Foliage: Length: 4-21 cm. Width: 1-4.5 cm. Shape: Lanceolate. Shape of Apex: Acuminate. Shape of Base: Attenuate to Acute. Orientation and aspect: Straight to curved. Texture: Upper surface: Rough. Lower surface: Rough. Young leaves: Upper surface color: NN137A. Lower surface color: 137B. Fully expanded leaves: Upper surface color: NN137A. Lower surface color: 137B. Venation: Type: Cross venulate to longitudinal. Color: Upper surface: 145A. Lower surface: 145B. Margin: Entire.
   Flowers.—Bud: Stage of opening: Single type blossom. Shape: Elliptic shaped ray florets and tubular disk floret. Diameter: 1-3.2 cm. Length: 1.2-2.3 cm. Color: When closed: NN137A. When open: Natural Flowering Season: 13-14 weeks after planting. Inflorescence: Form: Single flower. Disc and ray floret arrangement: Single composite flower. Average number per plant: 20-30. Average number per branch: 1-3. Fragrance: None. Diameter: 4-9 cm. Height: 2-3.5 cm. Diameter of disc: 1.5-3.5 cm. Receptacle height: 1 cm. Receptacle diameter: 1.5-3.5 cm. Ray florets: Number per inflorescence: 16-23. Arrangement: In two or three whorls. Length: 1.5-3.7 cm. Width: 0.9-1.3 cm. Shape: Ellipse. Shape of Apex: Obtuse. Shape of Base: Acute. Margin: Entire. Texture: Upper surface: Smooth, glabrous. Lower surface: Rough. Color: When opening: Upper surface: N45A. Lower surface: 53A. At maturity: Upper surface: N45A. Lower surface: 53A. Ground color description: Red. Disc florets: Number per inflorescence: Numerous, too many too count. Arrangement: In center of receptacle Length: 8-15 mm. Width: 1-2 mm. Shape: Tubular. Shape of Apex: Sharp. Color: Immature: 187A. At maturity: 187A. Phyllaries: Number per inflorescence: 35-50. Arrangement: In 304 whorls. Length: 0.5-1.5 cm. Width: 2-3 mm. Shape: Lanceolate. Shape of Apex: Elongate, oval. Shape of Base: Fused. Margin: Entire. Texture: Upper surface: Rough. Lower surface: Rough. Color: Immature: Upper surface: NN137B. Lower surface: NN137B. At maturity: Upper surface: NN137B. Lower surface: NN137B.
   Reproductive organs.—Androecium: Presence: On disk florets. Number: One. Filament: Length: 1-2 mm. Color: 187B. Anther: Shape: Oval. Length: 1 mm. Color: 187B. Pollen: Color: 15B. Amount: Too much to quantify. Gynoecium: Presence: In disk and ray florets. Pistil length: 1-2 mm. Stigma: Shape: Two parted. Color: 187A. Style: Length: 1-2 mm. Color: 187A. Seeds/fruit: Length: 1-2 mm. Color:
   Disease/pest resistance.—Disease resistance: No additional disease resistance has been observed beyond the typical resistance in Echinacea plants.
   Pest resistance.—No additional pest resistance has been observed beyond the typical resistance in Echinacea plants. Weather tolerance: Temperature tolerance: No additional temperature resistance has been observed beyond the typical resistance in Echinacea plants. Drought tolerance: No additional drought resistance has been observed beyond the typical resistance in Echinacea plants.

I claim:

1. A new and distinct cultivar of Echinacea plant named ‘BullEchipur 122’, as illustrated and described.
